(19) United States
(12) Patent Application Publication
(10) Pub. No.: US 2023/0165511 A1
(43) Pub. Date: Jun. 1, 2023

(54) COGNITIVE ABILITY DETECTION DEVICE AND COGNITIVE ABILITY DETECTION METHOD

(71) Applicant: Murata Manufacturing Co., Ltd., Nagaokakyo-shi (JP)

(72) Inventor: Shingo OKAJIMA, Nagaokakyo-shi (JP)

(21) Appl. No.: 18/161,917

(22) Filed: Jan. 31, 2023

Related U.S. Application Data
(63) Continuation of application No. PCT/JP2021/029519, filed on Aug. 10, 2021.

(30) Foreign Application Priority Data
Aug. 20, 2020 (JP) 2020-139206

(51) Int. Cl. A61B 5/375; A61B 5/398; A61B 5/372

(52) U.S. Cl. CPC A61B 5/375; A61B 5/398; A61B 5/372; A61B 2503/12; A63F 13/798

(57) ABSTRACT

A cognitive signal generation unit of a cognitive ability detection device includes a brain signal acquisition unit, a database, an MRCP correction data selection unit, and a calculation unit. The brain signal acquisition unit acquires a brain signal including an event-related potential. The database stores motor readiness potential correction data corresponding to an operation of a subject. The MRCP correction data selection unit selects the motor readiness potential correction data, on the basis of prior information including the type of an action, and outputs the motor readiness potential correction data to the calculation unit. The calculation unit generates the cognitive signal by correcting the brain signal with the motor readiness potential correction data.

### CROSS REFERENCE TO RELATED APPLICATION

This is a continuation of International Application No. PCT/JP2021/029519 filed on Aug. 10, 2021, which claims priority from Japanese Patent Application No. 2020-139206 filed on Aug. 20, 2020. The contents of these applications are incorporated herein by reference in their entireties.

### BACKGROUND ART

The present disclosure relates to a cognitive ability detection device and a cognitive ability detection method for detecting cognitive ability to a stimulus from the outside.

Patent Literature 1 describes a cognitive ability detection technique using a brain signal. The technique described in Patent Literature 1 detects an event-related potential from a brain signal and uses the event-related potential to detect cognitive ability.

Patent Literature 2 describes a technique for analyzing and diagnosing a brain cerebration function that uses brain wave data. The technique in Patent Literature 2 detects a motor readiness potential from brain wave data and uses the motor readiness potential to diagnose a brain cerebration function.

Patent Literature 3 describes a behavior prediction technique using a brain wave. The technique of Patent Literature 3 predicts a behavior of a person by using a motor readiness potential.

Patent Literature 1

Japanese Patent Unexamined Publication No. 2002-272692 bulletin

Patent Literature 2

Japanese Patent Unexamined Publication No. 2018-192909 bulletin

Patent Literature 3

International Publication WO 2020/138012 A

### BRIEF SUMMARY

However, in such a situation that a motor readiness potential as described in Patent Literatures 2 and 3 is generated, the event-related potential in the technique described in Patent Literature 1 includes a motor readiness potential together with an event-related potential (hereinafter, referred to as cognitive potential) such as the P300 generated at the time of recognition.

When such a motor readiness potential is present, the measurement accuracy of the cognitive potential may be deteriorated.

Therefore, an object of the present disclosure is to provide a technique for improving the measurement accuracy of the cognitive potential such as the P300.

A cognitive ability detection device of the present disclosure includes a brain signal acquisition unit, a correction data storage unit, and a cognitive signal generation unit. The brain signal acquisition unit acquires a brain signal including an event-related potential. The correction data storage unit stores motor readiness potential correction data depending on a type of an action. The cognitive signal generation unit generates a cognitive signal by correcting the brain signal with the motor readiness potential correction data.

This configuration suppresses the motor readiness potential included in the event-related potential (cognitive potential).

The present disclosure can improve the measurement accuracy of the cognitive potential.

### BRIEF DESCRIPTION OF THE DRAWINGS

FIG. 1 is a functional block diagram illustrating a configuration of a cognitive signal generation unit according to a first embodiment.

FIG. 2 is a diagram illustrating a configuration of a cognitive ability detection system according to the first embodiment.

FIGS. 3A, 3B, and 3C are tables illustrating examples of correction data stored in a database.

FIG. 4A is a diagram illustrating an example of a waveform of a brain signal, and FIG. 4B is an enlarged view of a region including the electro-oculogram (EOG) and the P300 in the waveform illustrated in FIG. 4A.

FIG. 5 is a diagram illustrating an example of the motor readiness potential correction data.

FIG. 6 is a diagram illustrating an example of a waveform of the cognitive signal.

FIG. 7 is a diagram illustrating an example of a waveform of the brain signal.

FIG. 8 is a diagram illustrating an example of the motor readiness potential correction data.

FIG. 9 is a diagram illustrating an example of a waveform of the cognitive signal.

FIG. 10 is a flowchart illustrating an example of a generation method of a database.

FIGS. 11A, 11B, 11C, and 11D are diagrams each illustrating an example of a video at the time of database generation.

FIG. 12 is a flowchart illustrating an example of a generation method of a cognitive signal.

FIGS. 13A, 13B, 13C, and 13D illustrate respective waveforms in a case where a plurality of actions are performed with respect to one cognition.

FIGS. 14A, 14B, 14C, and 14D illustrate respective waveforms in a case where a plurality of actions are individually performed on each of a plurality of consecutive cognitions.

FIG. 15 is a functional block diagram illustrating a configuration of a cognitive signal generation unit according to a second embodiment.

FIG. 16 is a diagram illustrating a configuration of a cognitive ability detection system according to the second embodiment.

FIG. 17 is a diagram illustrating part of a configuration of a cognitive ability detection system according to a third embodiment.

FIG. 18 is a diagram illustrating a configuration of a cognitive ability detection system for games.

FIG. 19 is a diagram illustrating a configuration of a cognitive ability detection system for a game in multiplay.

### DETAILED DESCRIPTION

(First embodiment)

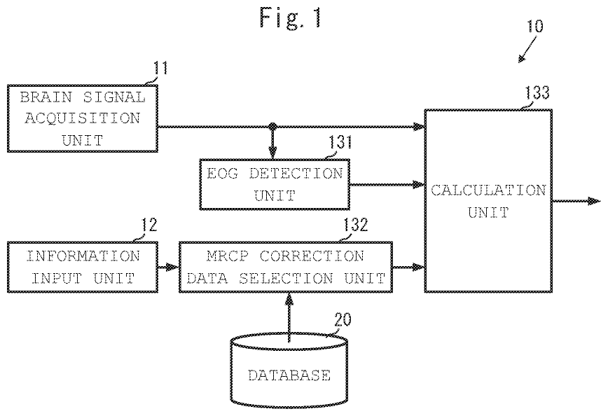

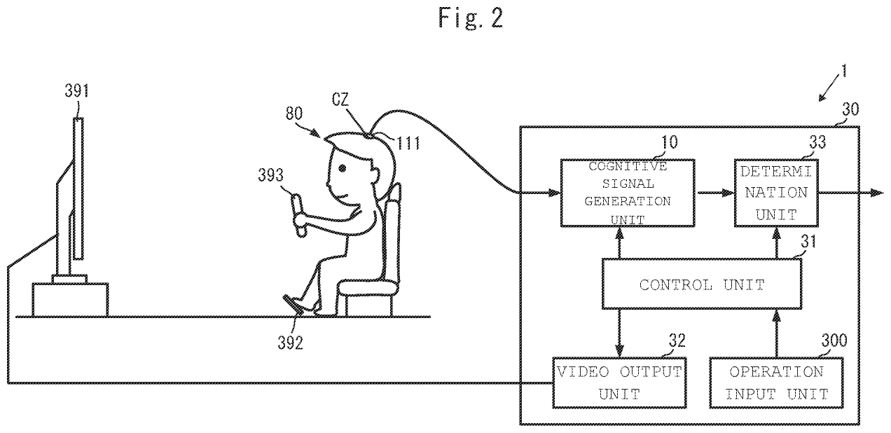

A cognitive ability detection device according to a first embodiment of the present disclosure will be described with reference to the drawings. FIG. 1 is a functional block diagram illustrating a configuration of a cognitive signal generation unit according to the first embodiment. FIG. 2 is a diagram illustrating a configuration of a cognitive ability detection system according to the first embodiment. In the present embodiment, a description will be made using as an example a case where a cognitive ability test is performed on driving. In other words, the present embodiment described an example in which the cognitive ability test is applied to a drive simulator.

(Configuration of Cognitive Ability Detection System 1)

As illustrated in FIG. 2, the cognitive ability detection system 1 includes: a cognitive ability detection device 30 including a cognitive signal generation unit 10; a brain signal sensor 111; a display 391; a simulated pedal 392; and a simulated steering wheel 393.

The display 391 is disposed in front of a subject 80. The simulated pedal 392 and the simulated steering wheel 393 are disposed at such positions that the subject 80 can operate the simulated pedal 392 and the simulated steering wheel 393. Note that, in FIG. 2, a specific (physical) configuration of the cognitive ability detection system 1 (drive simulator) is not illustrated except for the display 391, the simulated pedal 392, and the simulated steering wheel 393.

The brain signal sensor 111 is attached to the subject 80. More specifically, the brain signal sensor 111 is attached at a position including the top of the head (the position of CZ on a scalp potential distribution map (International 10-20 method)) of the subject 80.

The cognitive ability detection device 30 is connected to the brain signal sensor 111 and the display 391. The cognitive ability detection device 30 is implemented by an arithmetic processing unit or the like such as a processor (e.g., of a personal computer).

The cognitive ability detection device 30 includes the cognitive signal generation unit 10, a control unit 31, a video output unit 32, a determination unit 33, and an operation input unit 300.

The operation input unit 300 receives, from a user or the like, operation inputs such as: an input of a trigger for start and end of a cognitive ability detection test; and a selection of a type of the cognitive ability detection test, and the operation input unit 300 outputs the operation inputs to the control unit 31.

The control unit 31 performs overall control of the cognitive ability detection device 30. The control unit 31 controls the start, end, and the like of the cognitive ability detection test in accordance with the operation inputs from the operation input unit 300. Furthermore, the control unit 31 instructs the video output unit 32 to output a video for the selected cognitive ability detection test.

In addition, the control unit 31 outputs prior information corresponding to the selected cognitive ability detection test to the cognitive signal generation unit 10. The prior information corresponding to the cognitive ability detection test is information defining a type of an action or the like that the subject 80 causes in response to recognition of danger. For example, the prior information defines that, in response to cognition of a sudden appearance of a person, an operation of a brake pedal or a steering wheel is performed. Note that the prior information may include, for example, identification information of the subject 80, type information of the subject 80, and the like.

The video output unit 32 outputs a video of the selected cognitive ability detection test to display 391. The display 391 displays this video. As a result, the subject 80 can view the video of the cognitive ability detection test.

When the subject 80 views this video and operates the simulated pedal 392 and the simulated steering wheel 393, the brain signal (brain wave) includes an event-related potential. The brain signal sensor 111 detects the brain signal and outputs the brain signal to the cognitive signal generation unit 10.

Although a more specific configuration and processing will be described later, the cognitive signal generation unit 10 generates a cognitive signal from the brain signal detected by the brain signal sensor 111.

The determination unit 33 analyzes the cognitive signal to determine a cognitive ability, for example, to determine whether the subject 80 has a cognitive ability and to determine the level of the cognitive ability of the subject 80. Note that the determination of the cognitive ability by using the cognitive signal uses, for example, an appearance of the P300.

The determination of the cognitive ability can use various known methods, and therefore the description thereof will be omitted here.

(Configuration of Cognitive Signal Generation Unit 10)

As illustrated in FIG. 1, the cognitive signal generation unit 10 includes a brain signal acquisition unit 11, an information input unit 12, an EOG detection unit 131, an MRCP correction data selection unit 132, a calculation unit 133, and a database 20. The database 20 corresponds to the correction data storage unit of the present disclosure. The abbreviation MRCP is a movement-related cortical potential, and means a motor-related potential (motor readiness potential) in the present disclosure.

The brain signal acquisition unit 11 acquires a brain signal from the brain signal sensor 111 and outputs the brain signal to the calculation unit 133 and the EOG detection unit 131. The brain signal acquisition unit 11 may include an amplifier circuit and a filter circuit. By including an amplifier circuit, the brain signal acquisition unit 11 can amplify the brain signal to a predetermined signal level (amplitude). By including a filter circuit, the brain signal acquisition unit 11 can suppress noise components other than the event-related potential included in the brain signal.

The information input unit 12 is an input interface for prior information. The information input unit 12 receives the prior information from the control unit 31 described above, and outputs the prior information to the MRCP correction data selection unit 132. The information input unit 12 further includes a user interface, and may receive the prior information by an operation input from the outside. Note that the prior information from the control unit 31 may be directly input to the MRCP correction data selection unit 132. That is, the information input unit 12 can be omitted.

The EOG detection unit 131 detects an electro-oculogram EOG from the brain signal. The EOG detection unit 131 detects a saccade and a fixation from the electro-oculogram. The EOG detection unit 131 detects a timing at which a change from the saccade to the fixation, and outputs the timing of the change to the calculation unit 133 as a reference timing.

Note that the EOG detection unit 131 may output detection results of the saccade and the fixation to the calculation unit 133. In this case, the calculation unit 133 may detect the timing of the change from the saccade to the fixation and set the timing as the reference timing.

The database 20 stores correction data (motor readiness potential correction data) corresponding to the motor readiness potential for each action or for each subject. The correction data is data that simulates the waveform of the motor readiness potential corresponding to the action or the subject. These pieces of correction data are acquired by data sampling processing in advance (details will be described later) and stored in the database 20.

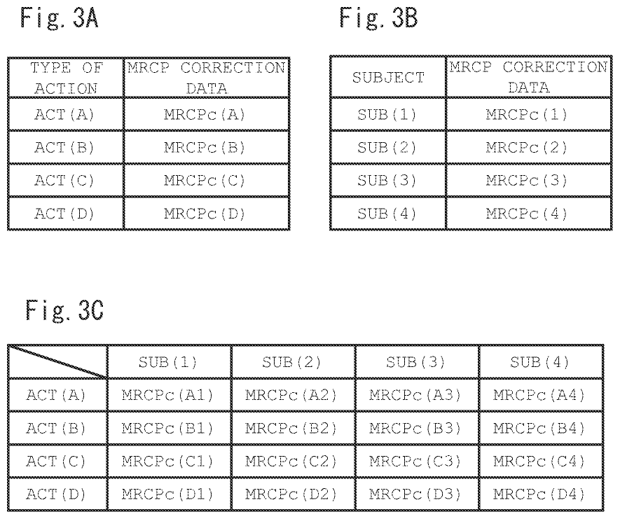

FIGS. 3A, 3B, and 3C are tables illustrating examples of correction data stored in the database. In each table of FIGS. 3A, 3B, and 3C, the motor readiness potential correction data is written as MRCP correction data.

In the case of FIG. 3A, the motor readiness potential correction data is set for each type of an action. For example, motor readiness potential correction data MRCPc(A), motor readiness potential correction data MRCPc(B), motor readiness potential correction data MRCPc(C), and motor readiness potential correction data MRCPc(D) are respectively set for an action ACT(A), an action ACT(B), an action ACT(C), and an action ACT(D). An actual example of each of the action ACT(A), the action ACT(B), the action ACT(C), and the action ACT(D) is, in the case of a drive simulator, a steering wheel operation, an accelerator operation, a brake operation, or the like under a specific environment.

In the case of FIG. 3B, the motor readiness potential correction data is set for each subject. For example, motor readiness potential correction data MRCPc(1), motor readiness potential correction data MRCPc(2), motor readiness potential correction data MRCPc(3), and motor readiness potential correction data MRCPc(4) are respectively set for a subject SUB(1), a subject SUB(2), a subject SUB(3), and a subject SUB(4).

In the case of FIG. 3C, the motor readiness potential correction data is set for each combination of the subject and the type of an action. Details of the individual combinations are not described, but, for example, the motor readiness potential correction data MRCPc(A1) is set for a combination of the action ACT(A) and the subject SUB(1), and the motor readiness potential correction data MRCPc(D4) is set for a combination of the action ACT(D) and the subject SUB(4).

The MRCP correction data selection unit 132 selects and reads out the motor readiness potential correction data stored in the database 20 by using the prior information from the information input unit 12. For example, if the action ACT(A) is specified in the prior information, the MRCP correction data selection unit 132 selects the motor readiness potential correction data MRCPc(A). If the subject SUB(2) is specified in the prior information, the MRCP correction data selection unit 132 selects the motor readiness potential correction data MRCPc(2). If the action ACT(A) and the subject SUB(2) are specified in the prior information, the MRCP correction data selection unit 132 selects the motor readiness potential correction data MRCPc(A2).

Note that the MRCP correction data selection unit 132 may select the motor readiness potential correction data with reference to a degree of importance. For example, in a case where the motor readiness potential correction data corresponding to a plurality of types of actions is stored, a degree of importance is associated with each action. When a plurality of types of actions are included in the prior information, the MRCP correction data selection unit 132 selects, for example, the motor readiness potential correction data corresponding to an action having the highest degree of importance.

The MRCP correction data selection unit 132 outputs the selected motor readiness potential correction data to the calculation unit 133.

The calculation unit 133 generates a cognitive signal by correcting the brain signal using the motor readiness potential correction data selected by the MRCP correction data selection unit 132 (which is selected correction data). More specifically, for example, the calculation unit 133 generates the cognitive signal by subtracting the selected correction data from the brain signal. At this time, the calculation unit 133 executes subtraction processing on the basis of the reference timing set by the EOG detection unit 131 or the calculation unit 133.

(Specific Generation Method of Cognitive Signal)

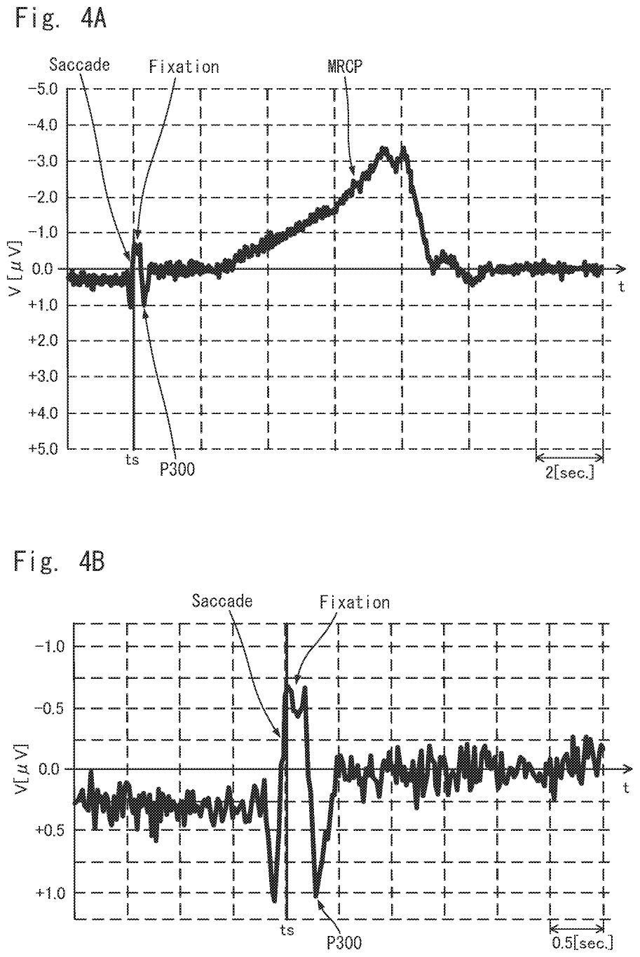

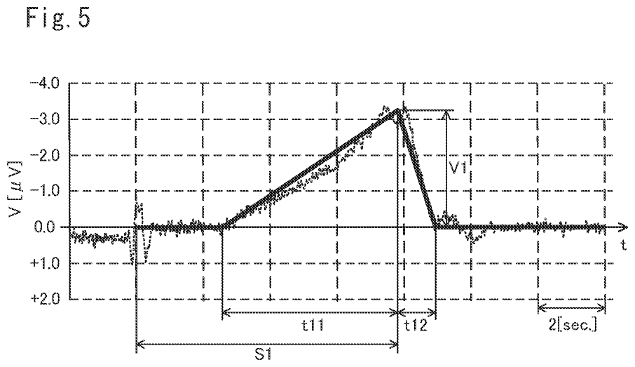

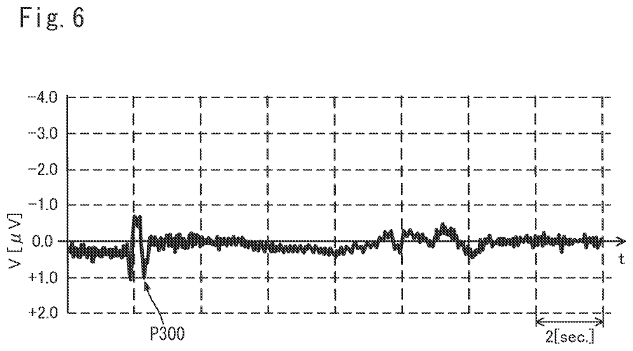

FIG. 4A is a diagram illustrating an example of a waveform of the brain signal, and FIG. 4B is an enlarged view of a region including the EOG and the P300 in the waveform illustrated in FIG. 4A. FIG. 5 is a diagram illustrating an example of the motor readiness potential correction data. FIG. 6 is a diagram illustrating an example of a waveform of the cognitive signal.

As illustrated in FIGS. 4A and 4B, the brain signal includes an electro-oculogram EOG including a saccade and a fixation, a cognitive event-related potential P300, and a motor readiness potential MRCP.

As illustrated in FIGS. 4A and 4B, the electro-oculogram EOG, the cognitive event-related potential P300, and the motor readiness potential MRCP each have a unique waveform (characteristic waveform). For example, the electro-oculogram EOG includes a saccade and a fixation. The saccade is generated by a movement of an eyeball caused by cognition, which rapidly changes (changes in a negative potential direction) a voltage due to the movement of the eyeball caused by cognition. The fixation stops the movement of the eyeball and gazes at a cognitive target, which stabilizes the voltage. The cognitive event-related potential P300 is a temporary voltage (temporary voltage in the positive potential direction) generated when the subject 80 cognizes an object, and is generated about 300 msec after the reference timing of cognition. The motor readiness potential MRCP is a voltage generated when the subject 80 performs an action in response to the cognition of cognitive target, and the voltage value gradually increases (negative potential) after the cognition, and the voltage value decreases (gets close to 0 V) with completion of the action.

As illustrated in FIG. 5, the motor readiness potential correction data is set on the basis of the motor readiness potential MRCP. By using the fact that the waveform of the motor readiness potential MRCP has the above characteristics, and the motor readiness potential correction data is set, for example, using a maximum voltage value V1, a time difference S1, a time difference t11, and a time difference t12 as illustrated in FIG. 5.

The maximum voltage value V1 is set by a maximum value (negative potential) of the motor readiness potential MRCP. The time difference Si is set by a time difference between the reference timing and a time of the maximum voltage value V1 (maximum value time). As described above, the reference timing is set by a timing of the change from the saccade to the fixation.

The time difference t11 is set by the time difference between the maximum value time and the time at which the motor readiness potential MRCP starts changing. The start time of the changing can be set as follows. For example, the motor readiness potential MRCP is subjected to approximation processing in such a manner that the voltage rise region is linear approximated, and the start time of the changing is set by the time at which the approximated line intersects with the 0-volt line. The setting of the start time of the changing does not need to be set in this manner.

The time difference t12 is set at the time difference between the maximum value time and the time at which the motor readiness potential MRCP ends changing. The end time of the changing can be set as follows. For example, the motor readiness potential MRCP is subjected to approximation processing in such a manner that the voltage drop region is linear approximated, and the start time of the changing is set by the time at which the approximated line intersects with the 0-volt line. The setting of the end time of the changing does not need to be set in this manner.

Note that these settings are performed on the basis of previously sampled potentials. The previous sampling may be performed in advance on a subject 80 him or herself, or it is possible to use a brain signal acquired at the time of a past cognitive ability test of the subject 80. Alternatively, these settings may also use a statistical value (for example, an average value, a median value, or the like) of the motor readiness potentials MRCP detected from a plurality of persons. In a case where the statistical value of the motor readiness potentials MRCP detected from a plurality of persons is used for settings, the settings may be performed in consideration of attributes such as gender and age of the subject 80.

In this manner, the motor readiness potential correction data is set by a plurality of numerical values that characterize the motor readiness potential MRCP. As a result, a storage capacity of the motor readiness potential correction data can be made small without suppressing the characteristics of the motor readiness potential MRCP.

As the motor readiness potential correction data, it is possible to use waveform data (all of the sampled voltage values) of the motor readiness potential MRCP sampled in advance.

The calculation unit 133 subtracts the motor readiness potential correction data set as described above from the brain signal by using, as a reference, the reference timing set by the EOG detection unit 131 or the calculation unit 133. At this time, by using linear interpolation or the like, the calculation unit 133 restores the waveform of the motor readiness potential correction data from the motor readiness potential correction data, as depicted by the solid line in FIG. 5. Then, the calculation unit 133 subtracts the waveform of the restored motor readiness potential correction data from the brain signal (waveform of the brain signal).

Here, the motor readiness potential correction data set as described above is similar to or substantially coincides with the motor readiness potential MRCP included in the brain signal acquired from the subject 80. Therefore, as illustrated in FIG. 6, the cognitive signal obtained by subtracting the motor readiness potential correction data from the brain signal is a signal in which the motor readiness potential MRCP is suppressed from the brain signal. In other words, the cognitive signal has a waveform in which the Electro-oculogram EOG and the cognitive event-related potential P300 appear more clearly.

As a result, the cognitive signal becomes a signal in which the cognitive ability can be detected more easily and more reliably. As a result, the measurement accuracy of the cognitive potential such as the P300 is improved. Then, the determination unit 33 can determine the cognitive ability with higher accuracy by using the cognitive signal.

Note that, in the above description, an aspect has been described in which the calculation unit 133 subtracts the motor readiness potential correction data from the brain signal without performing any other processing. However, the calculation unit 133 may subtract the motor readiness potential correction data from the brain signal after correcting the voltage values of the motor readiness potential correction data on the basis of a maximum voltage value of the acquired brain signal and a maximum voltage value of the motor readiness potential correction data. For example, the calculation unit 133 calculates a ratio between the maximum voltage value of the acquired brain signal and the maximum voltage value of the motor readiness potential correction data. The calculation unit 133 corrects the voltage values of the motor readiness potential correction data on the basis of this ratio and subtracts the corrected voltage values from the brain signal. As a result, the motor readiness potential included in the brain signal is more effectively suppressed.

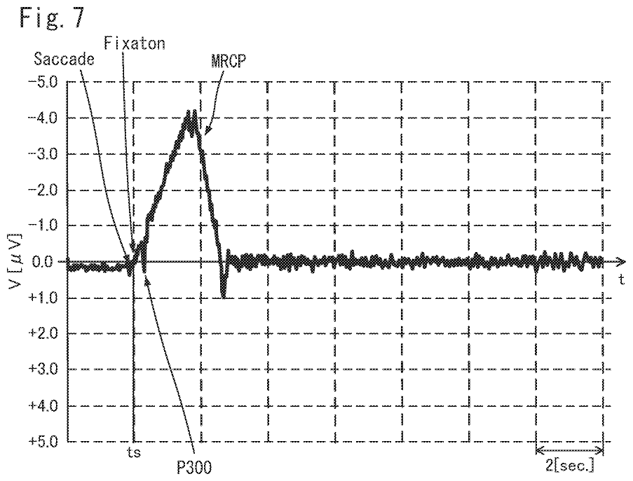

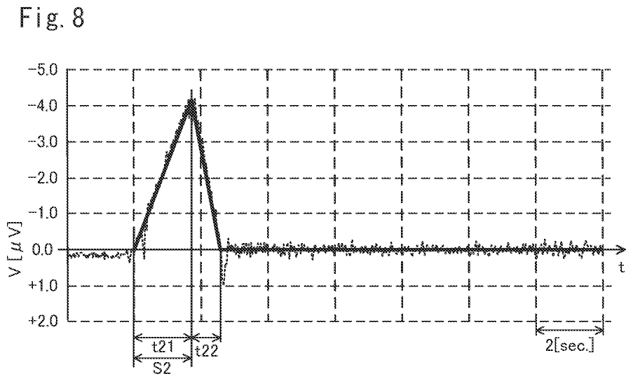

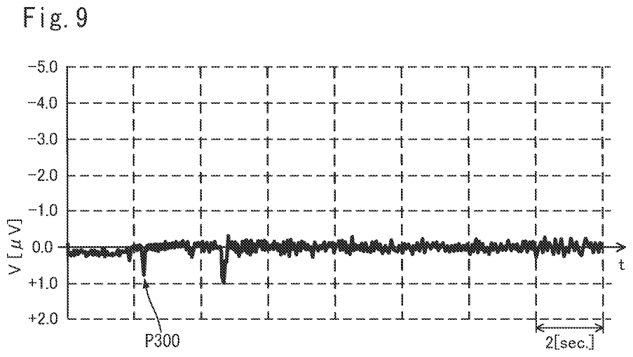

The above-described FIGS. 4A, 4B, 5, and 6 illustrate a case where a voltage change region of the motor readiness potential MRCP and the cognitive event-related potential P300 do not overlap each other. However, as illustrated in FIGS. 7, 8, and 9, even in a case where the voltage change region of the motor readiness potential MRCP and the cognitive event-related potential P300 overlap each other, by performing the above-described processing, the cognitive signal becomes a waveform in which the cognitive event-related potential P300 appears more clearly. FIG. 7 is a diagram illustrating an example of a waveform of the brain signal. FIG. 8 is a diagram illustrating an example of the motor readiness potential correction data. FIG. 9 is a diagram illustrating an example of the waveform of the cognitive signal.

As illustrated in FIG. 8, for an action or a subject whose motor readiness potential MRCP has a rapid voltage change, a motor readiness potential correction data (time difference S2, time difference t21, time difference t22) is set in accordance with the rapidness of the voltage change. Since an action or a subject are set as the prior information, the MRCP correction data selection unit 132 can select appropriate motor readiness potential correction data on the basis of the prior information.

Therefore, even when the waveform of the motor readiness potential correction data is different depending on actions or subjects, the cognitive signal has a waveform in which the cognitive event-related potential P300 appears more clearly as illustrated in FIG. 9. For example, even when the cognitive event-related potential P300 is buried in the motor readiness potential MRCP as illustrated in FIG. 7, the motor readiness potential MRCP is suppressed as illustrated in FIG. 9, and the cognitive event-related potential P300 can be easily detected.

(Generation Method of Database)

The motor readiness potential correction data stored in the above-described database 20 is generated as follows, for example.

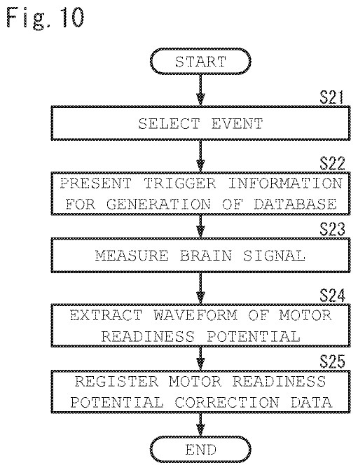

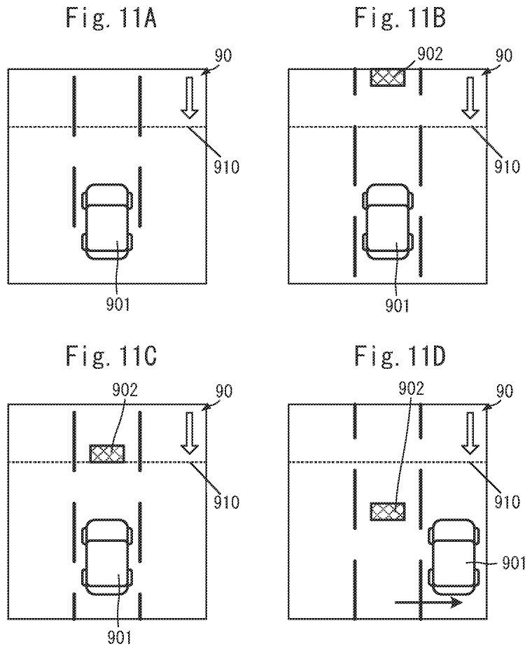

FIG. 10 is a flowchart illustrating an example of a generation method of the database. FIGS. 11A, 11B, 11C, and 11D are diagrams each illustrating an example of a video at the time of database generation.

First, a judge who judges the cognitive ability selects an event on which the cognitive ability will be determined (step S21). In other words, the cognitive ability detection device receives selection of an event.

The cognitive ability detection device presents trigger information corresponding to the selected event for generating a database, to a person such as a subject with respect to whom motor readiness potential correction data is to be generated (step S22). The trigger information for database generation is presented by, for example, a video as illustrated in FIGS. 11A, 11B, 11C, and 11D. Note that the trigger information is not limited to a video, and may be sound, stimulation, or the like.

In FIGS. 11A, 11B, 11C, and 11D, an automobile 901 and a reaction start line 910 are displayed in a video 90. The video 90 changes to move downward (see the thick arrows in the drawing) so that the automobile 901 moves upward on the video 90 while the automobile 901 does not change its position. At this time, a positional relationship between the automobile 901 and the reaction start line 910 does not change.

At a certain timing, as illustrated in FIG. 11(B), an avoidance target 902 appears from the upper end of the video 90. The person with respect to whom motor readiness potential correction data is to be generated has been given an instruction to start an avoidance action after the avoidance target 902 reaches the reaction start line 910. Therefore, in this state, the person with respect to whom motor readiness potential correction data is to be generated visually follows the avoidance target 902. As a result, an electro-oculogram EOG is generated.

Next, as illustrated in FIG. 11C, when the avoidance target 902 reaches the reaction start line 910, the person with respect to whom motor readiness potential correction data is to be generated operates the above-described simulated steering wheel so as to perform an avoidance action as illustrated in FIG. 11D. As a result, there are generated a cognition with respect to the avoidance action and a motor readiness potential for performing the avoidance motion.

The cognitive ability detection device measures (step S23) and acquires a brain signal in this series of actions.

The cognitive ability detection device extracts the waveform of the motor readiness potential from the brain signal (step S24). As described above, the start timing of the avoidance is roughly obtained from the video. Therefore, the cognitive ability detection device can more accurately extract the motor readiness potential by using as a reference the timing at which the avoidance target 902 set in the video reaches the reaction start line 910.

The cognitive ability detection device generates the above-described motor readiness potential correction data from the waveform of the extracted motor readiness potential, and registers the generated motor readiness potential correction data in the database 20 (step S25).

As described above, the database 20 of the motor readiness potential correction data can be generated by using the above-described method.

(Cognitive Ability Detection Method (Cognitive Signal Generation Method))

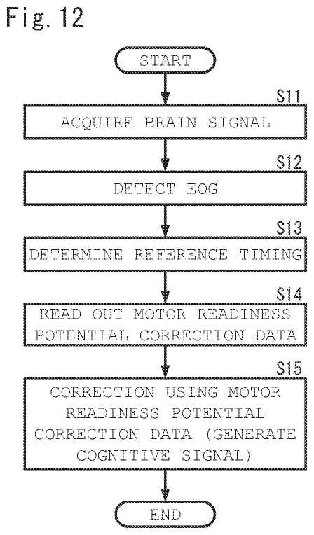

FIG. 12 is a flowchart illustrating an example of a generation method of a cognitive signal. The cognitive signal generation unit 10 generates the cognitive signal by performing the processing illustrated in FIG. 12. Note that details of each processing are described in the above description, and a description is omitted except for parts that require further additional description.

The cognitive signal generation unit 10 acquires a brain signal (step S11). The cognitive signal generation unit 10 detects an electro-oculogram EOG (step S12). The cognitive signal generation unit 10 determines a reference timing by using the electro-oculogram EOG (step S13).

The cognitive signal generation unit 10 reads a motor readiness potential correction data generated in advance as described above in correspondence to prior information (step S14). The cognitive signal generation unit 10 corrects the brain signal by using the read-out (selected) motor readiness potential data and generates a cognitive signal (step S15).

Note that, for example, this processing is programmed and stored in a storage medium, an external server, or the like. This processing can be implemented in such a manner that an arithmetic processing unit such as a processor (e.g., of a personal computer) that implements the cognitive signal generation unit 10 reads out and executes the program.

(Case where a Plurality of Actions Occur)

The above description describes a generation method of a cognitive signal in a case where one action occurs. However, there is a case where multiple actions occur in an overlapping manner or a continuous manner.

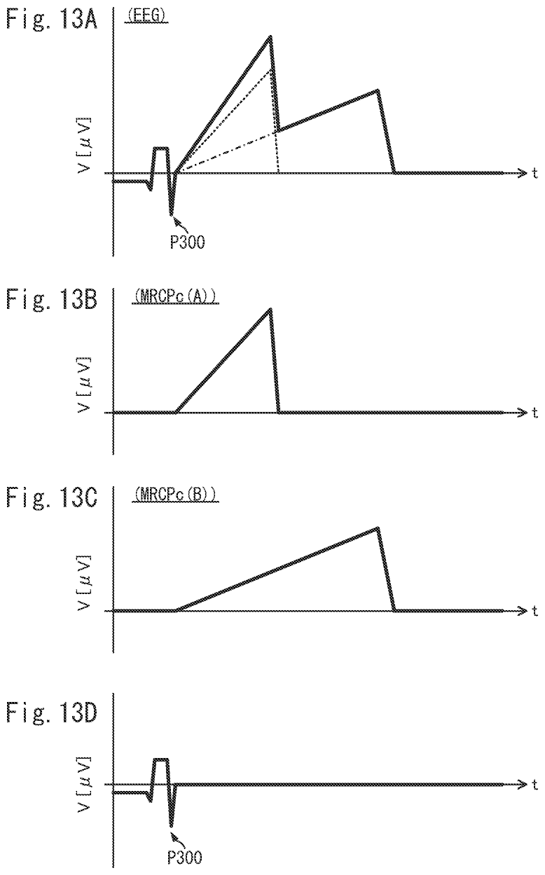

FIGS. 13A, 13B, 13C, and 13D illustrate respective waveforms in a case where a plurality of actions are performed with respect to one cognition. This case corresponds to, for example, a case where a pedestrian jumps out from a crosswalk, a brake pedal is stepped on, and a steering wheel is turned.

FIG. 13A illustrates a waveform of the brain signal, FIGS. 13B and 13C illustrate respective waveforms of the motor readiness potential correction data for different types of actions, and FIG. 13D illustrates a waveform of the cognitive signal.

As illustrated in the motor readiness potential correction data MRCPc(A) of FIG. 13B and the motor readiness potential correction data MRCPc(B) of FIG. 13C, the motor readiness potential correction data is set for each action. Therefore, as illustrated in FIG. 13A, even when a plurality of motor readiness potentials are included in the brain signal, each motor readiness potential can be suppressed. As a result, as illustrated in FIG. 13D, the cognitive signal becomes a signal in which the cognitive event-related potential P300 can be easily detected.

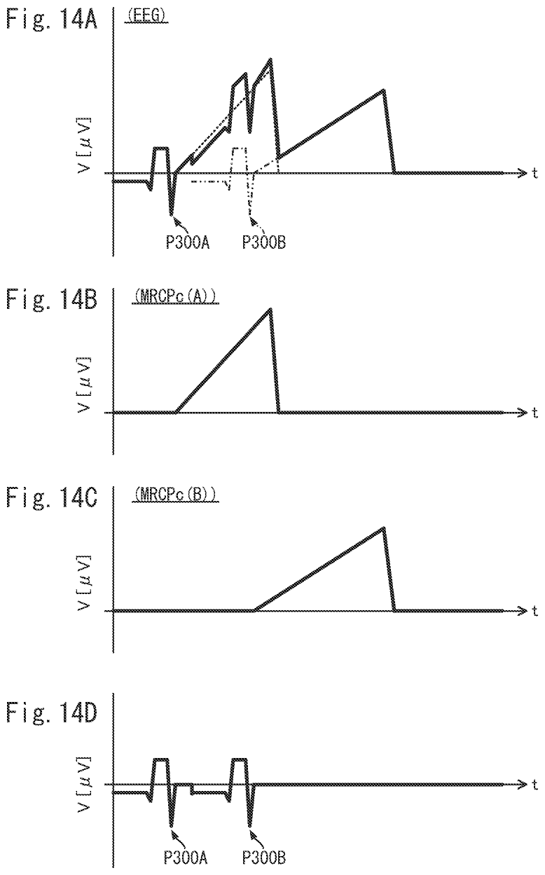

FIGS. 14A, 14B, 14C, and 14D illustrate respective waveforms in a case where a plurality of actions are individually performed on each of a plurality of consecutive cognitions. This case corresponds to, for example, a case where after a brake pedal is stepped on to slow down when getting close to a crosswalk, a steering wheel is turned in response to a pedestrian jumping out.

FIG. 14A illustrates a waveform of the brain signal, FIGS. 14B and 14C illustrate respective waveforms of the motor readiness potential correction data for different types of actions, and FIG. 14D illustrates a waveform of the cognitive signal.

As illustrated in the motor readiness potential correction data MRCPc(A) of FIG. 14B and the motor readiness potential correction data MRCPc(B) of FIG. 14C, the motor readiness potential correction data is set for each action. Therefore, as illustrated in FIG. 14A, even when a plurality of motor readiness potentials are included in the brain signal, each motor readiness potential can be suppressed. As a result, as illustrated in FIG. 14D, the cognitive signal becomes a signal in which a cognitive event-related potential P300A and a cognitive event-related potential P300B can be detected individually and easily.

(Second Embodiment)

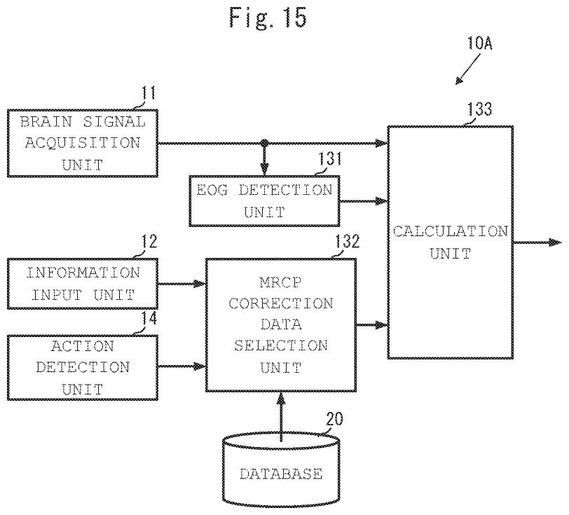

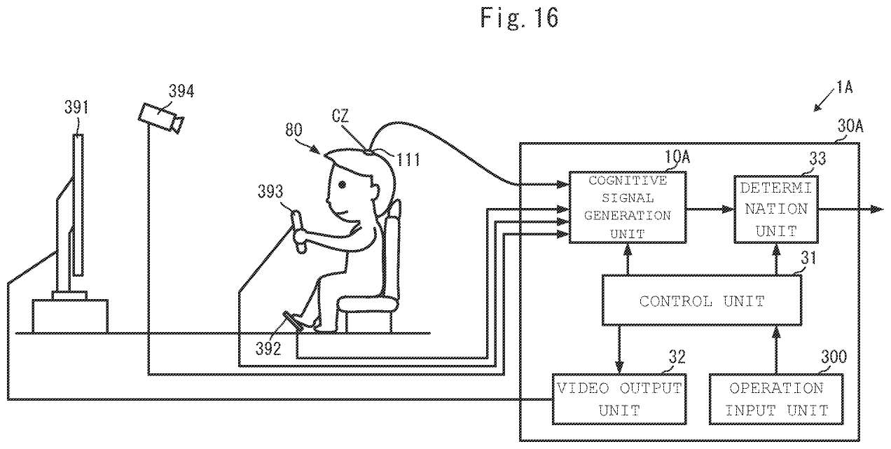

A cognitive ability detection device according to a second embodiment of the present disclosure will be described with reference to the drawings. FIG. 15 is a functional block diagram illustrating a configuration of a cognitive signal generation unit according to the second embodiment. FIG. 16 is a diagram illustrating a configuration of a cognitive ability detection system according to the second embodiment.

As illustrated in FIGS. 15 and 16, a cognitive ability detection system 1A according to the second embodiment is different from the cognitive ability detection system 1 according to the first embodiment in that a cognitive signal generation unit 10A in a cognitive ability detection device 30A includes an action detection unit 14 and in that a timing of an action detected by the action detection unit 14 is used. The other part of the configuration of the cognitive ability detection system 1A is similar to that of the cognitive ability detection device 30, and a description of a similar part will be omitted.

The cognitive ability detection system 1A includes a camera 394. For example, the camera 394 acquires a video including a body movement, a facial expression, a movement of the eye, and the like of a subject 80, and outputs an acquired image to the cognitive signal generation unit 10A. An action detection sensor such as an acceleration sensor or an angular velocity sensor is attached to a simulated pedal 392 and a simulated steering wheel 393. These action detection sensors detect a movement of the simulated pedal 392 (an operation of the subject 80) and a movement of the simulated steering wheel 393 (an operation of the subject 80), and output detected signals to the cognitive signal generation unit 10A. Note that means for mechanically detecting the movement of the simulated pedal 392 and the movement of the simulated steering wheel 393 may be provided, and the detected signals may be output based on mechanically detected results.

The action detection unit 14 of the cognitive signal generation unit 10A analyzes an eye movement and an action of the subject 80, and detects the types of the eye movement and the action, from the acquired video. Furthermore, the action detection unit 14 detects a type of the action (operation) of the subject 80 from the detected signals. The action detection unit 14 outputs the detected type of the action and the like to the MRCP correction data selection unit 132.

The MRCP correction data selection unit 132 selects motor readiness potential correction data on the basis of the type of the action detected by the action detection unit 14. As a result, the MRCP correction data selection unit 132 can select appropriate motor readiness potential correction data even without the prior information.

Alternatively, the MRCP correction data selection unit 132 can also select the motor readiness potential correction data on the basis of the detection result of the action detection unit 14 and the prior information. For example, when the detection result of the action detection unit 14 coincides with the prior information, the MRCP correction data selection unit 132 selects the motor readiness potential correction data on the basis of the type of a coincident action. When the detection result of the action detection unit 14 and the prior information do not coincide with each other, the MRCP correction data selection unit 132 selects the motor readiness potential correction data by using either the detection result or the prior information as a preferential reference. Alternatively, when the detection result of the action detection unit 14 and the prior information do not coincide with each other, the MRCP correction data selection unit 132 displays a warning indicating that there is no coincidence. As a result, for example, a judge who judges the cognitive ability may operate to input an appropriate type of an action into the cognitive ability detection device 30A.

Note that the detection result of the action detection unit 14 can also be used for generation of a cognitive signal in the calculation unit 133. For example, if the detection result of the action detection unit 14 includes an eye movement, the calculation unit 133 can set the reference timing by using the detection result of the action detection unit 14 even if the EOG detection unit 131 cannot detect the reference timing.

When the detection result of the action detection unit 14 includes an action (operation), the calculation unit 133 estimates a generation period of the motor readiness potential by using the timing of the action (operation). The calculation unit 133 performs correction by using the motor readiness potential correction data, during the estimated period in the brain signal. As a result, the calculation unit 133 can generate a cognitive signal from which the cognitive event-related potential P300 can be easily detected.

(Third Embodiment)

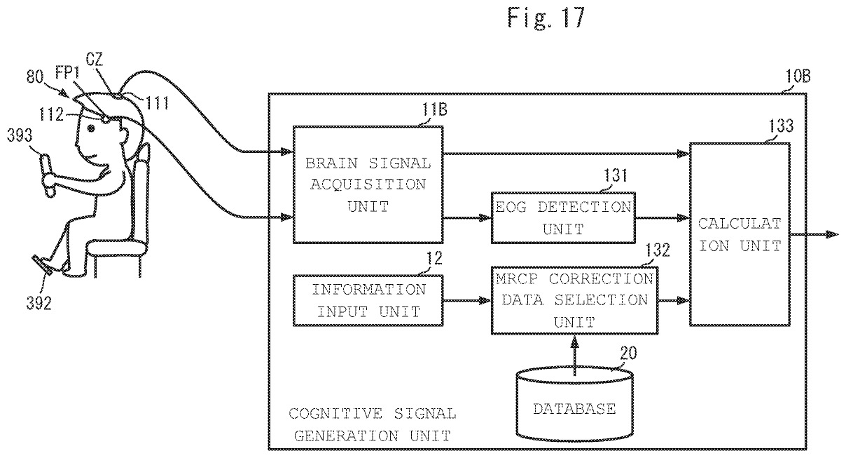

A cognitive ability detection device according to a third embodiment of the present disclosure will be described with reference to the drawings. FIG. 17 is a diagram illustrating part of a configuration of a cognitive ability detection system according to the third embodiment.

As illustrated in FIG. 17, the cognitive ability detection system according to the third embodiment is different from the cognitive ability detection system 1 according to the first embodiment in a configuration for detecting an EOG. The other part of the configuration of the cognitive ability detection system according to the third embodiment is similar to that of the cognitive ability detection system 1 according to the first embodiment, and a description of a similar part will be omitted.

A brain signal sensor 112 is attached to a subject 80. More specifically, the brain signal sensor 112 is attached at a position including the position of FP1 (International 10-20 method) of the subject 80. The brain signal sensor 112 outputs a detected brain signal to a brain signal acquisition unit 11B of a cognitive signal generation unit 10B.

The brain signal acquisition unit 11B outputs the brain signal (brain signal at CZ) detected by the brain signal sensor 111 to a calculation unit 133. The brain signal acquisition unit 11B outputs the brain signal (brain signal at FP1) detected by the brain signal sensor 112 to an EOG detection unit 131.

The EOG detection unit 131 detects an electro-oculogram EOG from the brain signal (brain signal at FP1) detected by the brain signal sensor 112.

With such a configuration, the brain signal serving as the detection source of the EOG is detected from the vicinity of an eye of the subject 80. Therefore, the EOG detection unit 131 can detect the EOG with higher accuracy.

In the above description, the P300 has been described as an example of the cognitive event-related potential. The cognitive event-related potential may be P100, N400, or the like, and by using the above-described configuration and processing, the cognitive signal generation unit can generate a cognitive signal from which these cognitive event-related potentials can be detected.

In addition, the above description takes, as an example, a case where a cognitive ability test is performed on driving. However, the above-described configuration and processing can be applied to any event as long as an action is performed in response to visual recognition of the event.

For example, by measuring the cognitive ability of the subject by using a game machine or the like, the present disclosure can be applied to a cognitive test for e-sports players and athletes and to a cognitive test for students at school.

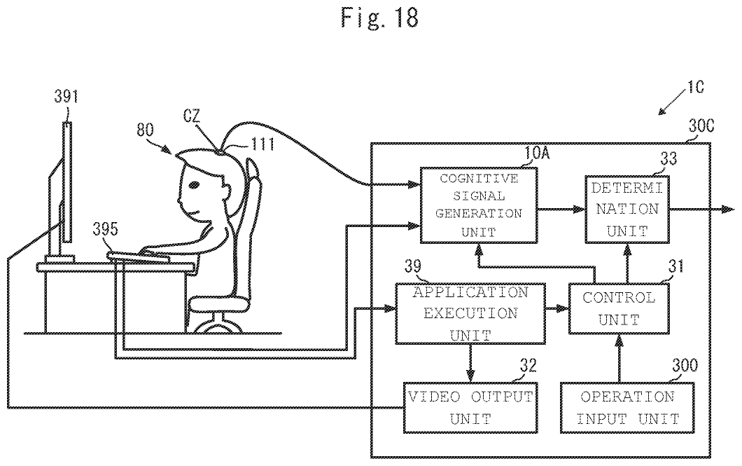

FIG. 18 is a diagram illustrating a configuration of a cognitive ability detection system for games. Hereinafter, a description will be given to a cognitive ability detection system 1C illustrated in FIG. 18 only about a part different from that of the cognitive ability detection system 1A according to the second embodiment.

As illustrated in FIG. 18, the cognitive ability detection system 1C includes a cognitive ability detection device 30C, a display 391, and an operation device 394.

The cognitive ability detection device 30C includes an application execution unit 39. The application execution unit 39 executes a game application.

The application execution unit 39 outputs a video of a game to the video output unit 32. The video output unit 32 outputs the video of the game to the display 391. As a result, the video of the game is displayed on the display 391.

The application execution unit 39 outputs, to the control unit 31, event information (a specific operation or the like to be input in correspondence to the game video) that can be used to detect the cognitive ability in the game.

On the basis of the event information, the control unit 31 outputs prior information corresponding to a cognitive ability detection test to the cognitive signal generation unit 10.

The operation device 394 is, for example, a keyboard, a mouse, or the like and receives an operation input of the subject 80 who is a game player. The operation device 394 outputs an operation input content to the cognitive signal generation unit 10 and the application execution unit 39.

The application execution unit 39 executes processing in the game application, in accordance with the operation input content.

The cognitive signal generation unit 10A detects the type of an action (operation) of the subject 80 by using the operation input content from the operation device 394.

Such a configuration enables the cognitive ability detection system 1C to detect the cognitive ability of the game player with respect to the game. Then, for example, the cognitive ability detection system 1C can detect such characteristics of the game player with respect to game operations that the game player themselves does not notice, from the detection result of the cognitive ability, and the game player can get feedback. Examples of the feedback method include visualized data of the cognitive ability and visualized data of a weak point (problem) based on the detection result of the cognitive ability. As a result, the game player can recognize his or her own weak point and can increase the speed of improving game skill.

Although FIG. 18 illustrates a case where the game machine is implemented by a personal computer (PC), the configuration of the present disclosure can be applied also to a console type game machine. In this case, the operation device 394 is not limited to a keyboard, and may be a controller dedicated to the game machine.

Furthermore, FIG. 18 illustrates a case where a general game application is used, but a test game application for detecting cognitive ability may be used. In this case, the test game application may be executed by the control unit 31.

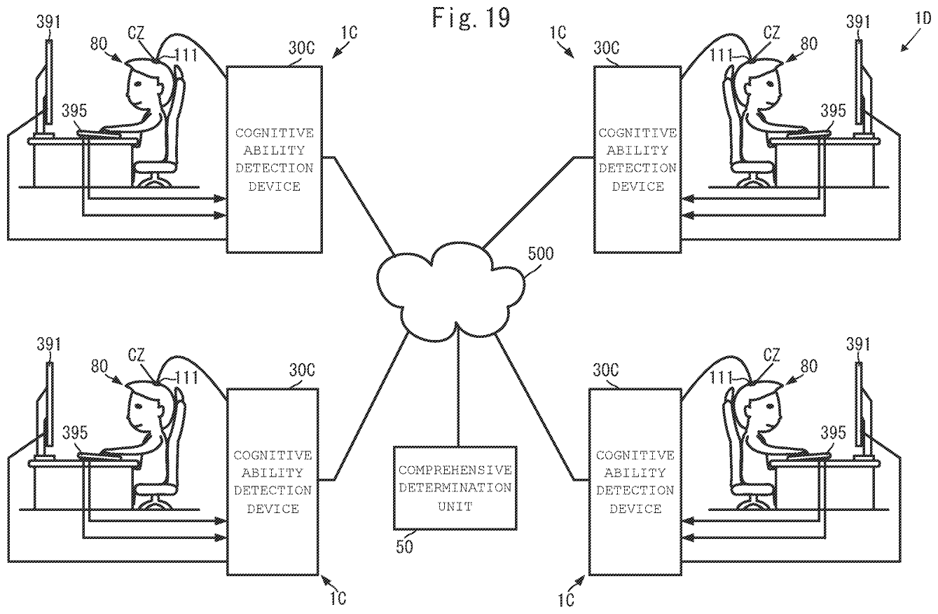

Furthermore, FIG. 18 illustrates the case of soloplay, but as illustrated in FIG. 19, the configuration of the present disclosure can be applied also to the case of multiplay.

FIG. 19 is a diagram illustrating a configuration of a cognitive ability detection system for a game in multiplay.

As illustrated in FIG. 19, a cognitive ability detection system 1D compatible to a multiplay environment includes a plurality of (four in FIG. 19) cognitive ability detection systems 1C, a comprehensive determination unit 50, and a data communication network 500.

The plurality of cognitive ability detection systems 1C are connected to the data communication network 500, and can transmit and receive data through the data communication network 500. The comprehensive determination unit 50 is connected to the data communication network 500 and acquires detection results of cognitive abilities, and various types of data and information for detecting cognitive abilities from the plurality of cognitive ability detection systems 1C.

The comprehensive determination unit 50 uses the detection results of the cognitive abilities from the plurality of cognitive ability detection systems 1C to determine the characteristics related to the cognitive ability in multiplay. For example, the comprehensive determination unit 50, from comparison results of the cognitive abilities of the plurality of players detected by the plurality of cognitive ability detection systems 1C, determines and visualizes a weak point or the like of the group in the case of multiplay.

At this time, the comprehensive determination unit 50 may use operation input contents to determine. For example, in the case of a cooperation play, the role of each player of the group is determined from the operation input content. The comprehensive determination unit 50 stores determination criteria for cognitive ability corresponding to each role, and determines cognitive abilities by using the determination criteria. As a result, it is possible to more accurately determined a weak point or the like when each player plays each role, and it is possible to provide the weak points and the like in a visual manner.

In addition, the comprehensive determination unit 50 stores characteristics of cognitive abilities suitable for each role in a cooperation play. Furthermore, the comprehensive determination unit 50 may determine an appropriate role on the basis of the acquired cognitive ability of each player, and can provide the roles in a visual manner. As a result, each player of the group performing a cooperation play can play the game, playing a role more suitable for the game. Therefore, for example, it is possible to try a quest game or the like having a higher level of difficulty, and motivation for the cooperation play can therefore be raised.

In the case of a battle play, an operation of each player (attack, defense, or the like) in a battle is determined from operation input contents. The comprehensive determination unit 50 stores a determination criterion for cognitive ability corresponding to each operation, and determines the cognitive ability by using the determination criterion. As a result, when each player fights against the opponent player, it is possible to more accurately determine a point in which each player is inferior to the opponent player, in other words, a weak point or the like at the time of the battle play, and the weak point can be provided in a visual manner. At this time, the comprehensive determination unit 50 can detect not only the cognitive ability but also a reaction speed of operation and the like by using the detection timing of the operation input content. The comprehensive determination unit 50 can also determine a weak point by using such a reaction speed of operation and the like and can also provide the weak point in a visual manner.

### REFERENCE SIGNS LIST

1, 1A, 1C, 1D cognitive ability detection system

10, 10A, 10B cognitive signal generation unit

11, 11B brain signal acquisition unit

12 information input unit

14 action detection unit

20 database

30, 30A, 30C cognitive ability detection device

31 control unit

32 video output unit

33 determination unit

39 application execution unit

80 subject

90 video

111 brain signal sensor

112 brain signal sensor

131 EOG detection unit

132 MRCP correction data selection unit

133 calculation unit

300 operation input unit

391 display

392 simulated pedal

393 simulated steering wheel

394 camera

901 automobile

902 avoidance target

910 reaction start line

1. A cognitive ability detection device comprising:
at least one processor and memory configured to:
acquire a brain signal including an event-related potential;
store motor readiness potential correction data corresponding to an operation of a subject; and
generate a cognitive signal by correcting the brain signal with the motor readiness potential correction data.

2. The cognitive ability detection device according to claim 1, wherein when the action includes a plurality of types of actions, the at least one processor is configured to correct the brain signal by using the motor readiness potential correction data for each of the plurality of types of actions.

3. The cognitive ability detection device according to claim 1,
wherein the at least one processor is further configured to detect an electro-oculogram from the brain signal, and
wherein the at least one processor is configured to correct the brain signal by using the electro-oculogram as a reference.

4. The cognitive ability detection device according to claim 3, wherein the at least one processor is configured to correct the brain signal by using as a reference a timing at which a change from a saccade to a fixation occurs on the electro-oculogram.

5. The cognitive ability detection device according to claim 1,
wherein the at least one processor is further configured to select the motor readiness potential correction data by using prior information including the type of the action that is previously set, and
wherein the at least one processor is configured to correct the brain signal using the selected motor readiness potential correction data.

6. The cognitive ability detection device according to claim 5,
wherein the at least one memory is further configured to store the motor readiness potential correction data and an associated degree of importance, and
wherein the at least one processor is configured to select the motor readiness potential correction data by referring to the degree of importance.

7. The cognitive ability detection device according to claim 5,
wherein the at least one processor is further configured to detect an action of a subject emitting a brain signal, and
wherein the at least one processor is configured to select the motor readiness potential correction data by using the detected action.

8. The cognitive ability detection device according to claim 7, wherein the at least one processor is configured to correct the brain signal by referring to a timing of the detected action.

9. The cognitive ability detection device according to claim 1, wherein the at least one processor is further configured to determine cognitive ability by using the cognitive signal.

10. The cognitive ability detection device according to claim 1, wherein the at least one processor is further configured to output a video for determining cognitive ability.

11. A cognitive ability detection device comprising:
at least one processor and memory configured to:
acquire a brain signal including an event-related potential;
store motor readiness potential correction data corresponding to a type of an action; and
generate a cognitive signal by correcting the brain signal with the motor readiness potential correction data,
wherein the at least one memory is configured to store the motor readiness potential correction data by using, as the motor readiness potential correction data:
a maximum value of a voltage;
a time difference between a reference timing and a time of the maximum value of the voltage;
a time difference between the time of the maximum value of the voltage and a start time of a change in the voltage; and
a time difference between the time of the maximum value of the voltage and an end time of the change in the voltage, and
wherein the at least one processor is further configured to restore a voltage waveform from each of the stored maximum value of the voltage and the stored time differences.

12. A cognitive ability detection method comprising:
acquiring a brain signal including an event-related potential;
correcting the brain signal using a motor readiness potential correction data corresponding to an operation of a subject; and
generating a cognitive signal based on the corrected brain signal.

13. The cognitive ability detection device according to claim 1, wherein the operation is an action that the subject causes in response to recognition of danger.

14. The cognitive ability detection method according to claim 12, wherein the operation is an action that the subject causes in response to recognition of danger.

15. The cognitive ability detection device according to claim 7, wherein the at least one processor configured to detect an action of a subject emitting a brain signal is an accelerometer or an angular rate sensor.

16. The cognitive ability detection method according to claim 12, further comprising:
detecting an electro-oculogram from the brain signal,
wherein the brain signal is corrected by using the electro-oculogram as a reference.

17. The cognitive ability detection method according to claim 13, wherein the brain signal is corrected by using as a reference a timing at which a change from a saccade to a fixation occurs on the electro-oculogram.

18. The cognitive ability detection method according to claim 12, further comprising:
selecting the motor readiness potential correction data by using prior information including the type of the action that is previously set,
wherein the brain signal is corrected using the selected motor readiness potential correction data.

19. The cognitive ability detection method according to claim 18,
wherein the motor readiness potential correction data is selected by referring to a degree of importance associated with the motor readiness potential correction data.

20. The cognitive ability detection method according to claim 18, further comprising:
detecting an action of a subject emitting a brain signal,
wherein the motor readiness potential correction data is selected by using the detected action.
